(19) United States
(12) Patent Application Publication
(10) Pub. No.: US 2010/0010478 A1
(43) Pub. Date: Jan. 14, 2010

(54) INTRAURETHRAL CATHETER

(76) Inventor: Israel NISSENKORN, Tel Aviv (IL)

Correspondence Address: THE NATH LAW GROUP, 112 South West Street, Alexandria, VA, 22314, US

(21) Appl. No.: 12/493,772

(22) Filed: Jun. 29, 2009

Related U.S. Application Data
(60) Provisional application No. 61/129,651, filed on Jul. 9, 2008.

(51) Int. Cl. A61M 25/04

(52) U.S. Cl. 604/544

(57) ABSTRACT

A self-retaining intraurethral catheter (IUC) made of a hollow tube and comprising a proximal portion, a distal portion, a central tubular hollow portion therebetween, a proximal crown formed at said proximal portion, a first and a second distal crowns formed at said distal portion, said first crown being disposed closer to the central portion than said second crown and being spaced from said second crown by an inter-crown portion, at least said inter-crown portion being formed so as to allow shortening the device by the detachment of the second crown therefrom.

### FIELD OF THE INVENTION

This invention relates to a self-retaining intraurethral catheter for use as an indwelling catheter.

### BACKGROUND OF THE INVENTION

Bladder outlet obstruction is a frequent urological problem in aging men with benign prostatic hyperplasia (BPH) or prostate carcinoma. The enlarged prostate, constricts the prostatic urethra and bladder outlet thus causing voiding difficulties. The prostatic urethra is the proximal part of the urethra surrounded by the prostate gland.

Bladder outlet obstruction is associated with a plurality of undesirable symptoms such as difficulties in urination: a reduced urinary stream, an increased desire to urinate (urgency), frequent urination (frequency) or the complete inability to urinate (urinary retention) which may lead eventually to severe renal disorders. Surgery may frequently relieve the symptoms, but many patients cannot have surgery (e.g., men with metastatic prostate carcinoma), are unfit for surgery or are unwilling to undergo an operation for different reasons. To eliminate prostatic urinary symptoms symptoms, medical devices that attempt to maintain an open passageway through the prostatic urethra have been developed.

One of such medical devices is described in U.S. Pat. No. 4,973,301, and its description including the description of its functioning principle in the above reference is incorporated herein by reference. U.S. Pat. No. 4,973,301 describes a self-retaining intraurethral catheter (IUC) for indwelling implantation, made of semi-rigid plastic material, and being in the form of a tube having a central, hollow cylindrical portion and proximal and distal crowns of larger diameters than the central portion. These crowns are formed by the provision of axial slits in the tube and its compression in the area of the slits and heat setting in the compressed state. When in use, the proximal crown of the IUC is adapted to be retained in the bladder neck and the distal crown is adapted to engage the prostatic urethra and prevent proximal displacement of the IUC in the urethra. The IUC may be manufactured using two different semi-rigid plastics, each having different degree of flexibility. One example suggested in this connection in U.S. Pat. No. 4,973,301 is producing the tube's distal portion of more flexible polyurethane than its proximal portion, thereby reducing the friction between the IUC and urethra, when the IUC is inserted therein.

### SUMMARY OF THE INVENTION

In accordance with one aspect of the present invention there is provided an indwelling self-retaining intra-urethral catheter (IUC) for relief of urinary retention and essentially full continence, the length of which may be adjusted before its implantation to make it suitable for a particular patient in accordance with the lengths of the prostatic urethra.

The length of the IUC in general and intermediate tubular portion in particular depends on the anatomy of an individual in whom it is to be inserted, namely, based on the urethral length between the bladder neck and verumontanum. Typically, the length of the IUC ranges from 35 mm to 75 mm. The diameter of the IUC ranges between 14 and 16 French.

In particular, the IUC according to this aspect of the invention comprises at its proximal portion, a proximal crown, and at its distal portion two crowns, a first distal crown and a second distal crown spaced apart by a distal inter-crown portion extending between the first and second distal crowns.

In accordance with another aspect of the invention there is provided an IUC having at least one layer of coating comprised of a biocompatible, inert material, to prevent the adhesion of ionic or salt substance, such as calcium, and organic deposits like bacteria, blood clots, fats etc. or a combination thereof typically present in urine.

The layer may cover the IUC about its entire perimeter and length or, may be disposed only about a part thereof. This part should be the part in continuous contact with urine.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1 is a schematic perspective view of an IUC according to an example of the present invention;

FIG. 2 schematically illustrates one manner of using the IUC of FIG. 1;

FIG. 3 schematically illustrates another manner of using the IUC of FIG. 1

### DETAILED DESCRIPTION OF EMBODIMENTS

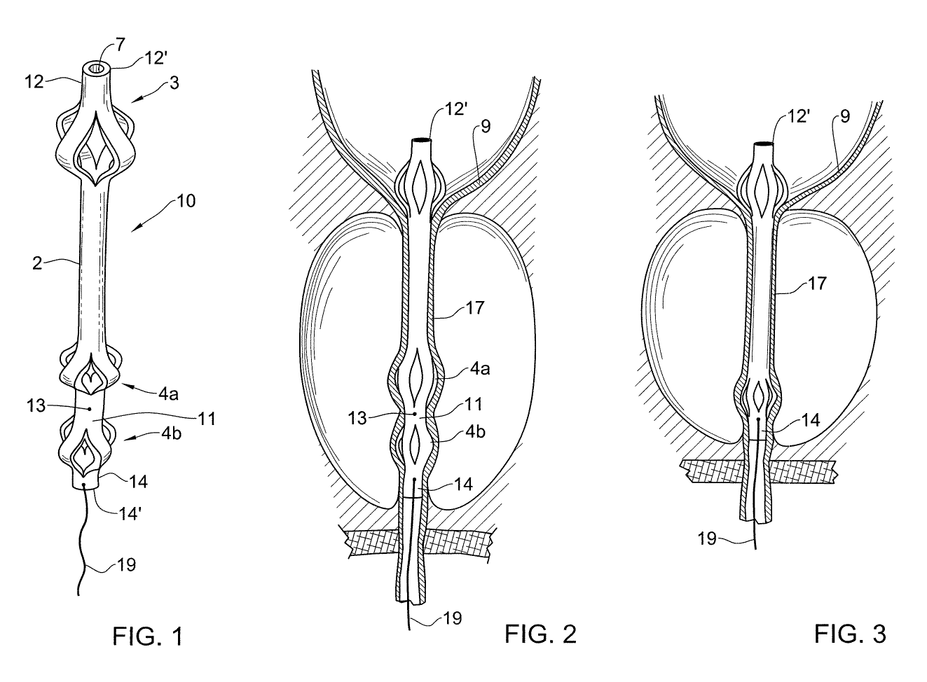

With reference to FIG. 1, a self-retaining IUC device 10, according to an example of the present invention, is shown adapted for being removably introduced into the urethra of a patient and for staying therein for a predetermined period of time.

The device 10 is in the form of a hollow tube with a proximal portion 12 associated with its proximal end 12′, a distal portion 14 associated with a distal end 14′, and a central portion 2 extending therebetween.

The central portion 2 is of a cylindrical form and has a hollow 7 allowing a flow passageway through device 10. The proximal portion 12 is formed with a proximal crown 3, and the distal portion 14 is formed with a first distal crown 4a, a second distal crown 4b and a distal inter-crown portion 11 extending between the first and second distal crowns 4a and 4b.

The distal crowns 4a and 4b have identical shapes and dimensions and they differ at least in dimensions from the proximal crown 3.

In particular, the external diameter of the proximal crown 3 is larger than that of the distal crowns 4a and 4b, and they all are greater than the external diameter of the central portion 2. The central portion may have an external diameter in the range of 4.5 and 5.5 mm. In this case, the external diameter of the proximal crown 3 may be in the range of 17-18 mm and the external diameter of the distal crowns 4a and 4b may be in the range of 12-13 mm. The length of the central portion 2 may be in the range of 10-30 mm.

The axial dimension of the proximal crown 3 is larger than that of the first and second distal crowns 4a and 4b. The axial dimension of the proximal crown 3 may be in the range of 11-15 mm and the axial dimensions of the distal crowns 4a and 4b may be in the range of 7-15 mm.

The device 10 is further formed with means facilitating its removal from the urethra after use. This means may be in the form of a 4/0 nylon stitch 19 attached to the device 10 at its distal portion 14 at a location 13 thereof either between the first and the second distal crowns 4a and 4b, or between the first distal crown 4a and the central portion 2.

The IUC is made of a semi-rigid plastic and is capable of being cut at least at the distal tubular portion 11 to allow shortening the device by cutting off the second crown 4b, when it is desired to shorten the length of the device, thus suiting it to the urethra of a specific patient. One example of a material from which the device 10 may be made is polyurethane marketed by BARD Angiomed under the trade name of Puroflex-Urosoft™.

The device 10 may be made from two different semi-rigid materials each having a different degree of flexibility. In particular, its distal portion 14 may be made of more flexible polyurethane than its proximal portion 12 to accommodate the urethral curvature and prevent the friction between the IUC 10 and urethra and reduce the chances for irritation or infection.

The device 10, and particularly its crowns 3, 4a and 4b may be produced by a process as described in U.S. Pat. No. 4,973,301, and the description of this process is incorporated herein by reference.

The device may be used for the insertion into a relatively long and a relatively short urethra, as illustrated in FIGS. 2 and 3, respectively. In all cases, the device 10 is inserted in the urethra and advances therethrough until its proximal end 12′ enters the bladder 8 and the proximal crown 3 is disposed in the bladder neck 9 preventing distal displacement of the device 10.

When the device 10 is used as shown in FIG. 2, the device 10, both its first and second distal crowns 4a and 4b press against the prostatic urethral inner wall 17 and prevent proximal displacement of the IUC.

When the device 10 is used as shown in FIG. 3, the distal end 14 thereof with the second distal crown 4b is cut off prior to the insertion of the device into the urethra, the crown sill prevents proximal IUC dislocation.

The existence of two distal crowns 4a and 4b in the device 10 allows a physician to use IUC for patients having urethra of different length by merely shortening the device. Moreover, when both distal crowns are used, the migration rate of the device 10 within urethra may be essentially reduced.

In accordance with another example of the present invention there is provided an IUC having at least one layer of coating comprised of a biocompatible, inert material, to prevent the adhesion thereto of ionic or salt substance(s), such as calcium, sodium etc., and organic deposits such as blood clots, fats etc. or a combination thereof. Such material may be for example polyHEMA substance or parylene. The layer may cover the IUC about its entire perimeter and length or, may be disposed only about a part thereof. This part should be the part in continuous contact with urine.

Those skilled in the art to which this invention pertains will readily appreciate that numerous changes, variations, and modifications can be made without departing from the scope of the invention, mutatis mutandis.

1. A self-retaining intraurethral catheter (IUC) made of a hollow tube and comprising a proximal portion, a distal portion, a central tubular hollow portion therebetween, a proximal crown formed at said proximal portion, a first and a second distal crowns formed at said distal portion, said first crown being disposed closer to the central portion than said second crown and being spaced from said second crown by an inter-crown portion, at least said inter-crown portion being formed so as to allow shortening the device by the detachment of the second crown therefrom.

2. An IUC according to claim 1, wherein at least said inter-crown portion is made of a semi rigid plastic material.

3. An IUC according to claim 2, wherein at least said inter-crown portion is made of a polyurethane.

4. An IUC according to claim 2, wherein at least the distal portion of the IUC is made of a semi rigid plastic material.

5. An IUC according to claim 1, wherein said first and second distal crowns are identical in shape and dimensions and have smaller axial and radial dimensions than those of the proximal crown.

6. An IUC according to claim 1, wherein at least one layer of coating comprised of a biocompatible, inert material is applied at least over a part thereof.

7. An IUC according to claim 6, wherein the coating is applied over the proximal portion thereof.

8. An IUC according to claim 6, wherein the at least one layer of coating comprises polyHEMA substance.

9. A self-retaining intraurethral catheter (IUC) made of a hollow tube and comprising a proximal portion, a distal portion, a central tubular hollow portion therebetween, a proximal crown formed at said proximal portion and at least one distal crown formed at said distal portion.

10. An IUC according to claim 9, wherein at least one layer of coating comprised of a biocompatible, inert material is applied at least over a part thereof.

11. An IUC according to claim 10, wherein the coating is applied over the proximal portion thereof.

12. An IUC according to claim 10, wherein the at least one layer of coating comprises polyHEMA substance.
